(19) United States
(12) Patent Application Publication
(10) Pub. No.: US 2017/0266044 A1
(43) Pub. Date: Sep. 21, 2017

(54) OXYGENATED OCULAR REGION TREATMENT METHODS, SYSTEMS, AND DEVICES

(71) Applicant: Roccor, LLC, Longmont, CO (US)

(72) Inventors: Mark S. Lake, Erie, CO (US); Kathryn Pate, Longmont, CO (US); William Brad Hensley, Superior, CO (US); Philip Keller, Berthoud, CO (US)

(21) Appl. No.: 15/460,509

(22) Filed: Mar. 16, 2017

Related U.S. Application Data
(60) Provisional application No. 62/308,960, filed on Mar. 16, 2016.

(51) Int. Cl. A61F 9/00; A61M 11/02

(52) U.S. Cl. CPC A61F 9/0026; A61M 11/02

(57) ABSTRACT

Methods, systems, and device for oxygenated ocular region treatment are provided. For example, a method of ocular region treatment is provided in accordance with various embodiments where an oxygenated material may be applied to an ocular region. The ocular region may include at least corneal tissue, limbal tissue, or ocular adnexal tissue. The oxygenated material may include at least an oxygenated emulsion, an oxygenated ointment, or an oxygenated liquid, which may be supersaturated in some cases. The oxygenated material may include perfluorocarbon, such as perfluorodecalin. The oxygenated material may include at least an antibiotic or an anesthetic in some cases. Some embodiments include an ocular region treatment system or device that may include an eye cup configured to surround an ocular region. A dispenser may be configured to couple with the eye cup and to dispense an oxygenated material to the ocular region may be provided.

### CROSS-REFERENCES TO RELATED APPLICATIONS

This application is a non-provisional patent application claiming priority benefit of U.S. provisional patent application Ser. No. 62/308,960, filed on Mar. 16, 2016 and entitled “OXYGENATED OCULAR TREATMENT METHODS, SYSTEMS, AND DEVICES,” the entire disclosure of which is herein incorporated by reference for all purposes.

### BACKGROUND

A growing percentage of battlefield trauma injuries may be occurring to the eyes. Throughout the wars in Afghanistan and Iraq, for example, ocular trauma may have resulted in more than 197,000 ambulatory patients and more than 4,000 hospitalizations. Traumatic eye injury may now rank fourth in terms of common injuries among active duty personnel. There may be widening gap in the treatment of ocular trauma, which may be primarily due to the continued use of antiquated treatment protocols and/or a lack of therapies that may be administered by people with limited medical training.

There may be a need for new tools and techniques to address ocular trauma, from the field to the hospital. Furthermore, there may be a need for new tools and techniques for ocular region treatment in general.

### SUMMARY

Methods, systems, and device for oxygenated ocular region treatment are provided in accordance with various embodiments. For example, a method of ocular region treatment is provided in accordance with various embodiments where an oxygenated material may be applied to an ocular region. The ocular region may include at least corneal tissue or limbal tissue, for example. In some embodiments, the ocular region includes ocular adnexal tissue and skin adjacent to the orbital cavity.

The oxygenated material may include at least an oxygenated emulsion, an oxygenated ointment, an oxygenated hydrogel, or an oxygenated liquid. The oxygenated emulsion, the oxygenated ointment, oxygenated hydrogel, or the oxygenated liquid may include at least a supersaturated-oxygenated emulsion, a supersaturated-oxygenated ointment, a supersaturated-oxygenated hydrogel, or a supersaturated-oxygenated liquid.

In some embodiments, the oxygenated material includes perfluorocarbon. The perfluorocarbon may include perfluorodecalin. The oxygenated material may include at least an antibiotic, anti-inflammatory, or an anesthetic.

In some embodiments, the oxygenated material is configured to produce a partial pressure of O₂ above that which exists at ambient atmospheric pressure when applied to the ocular region.

Some embodiments of the method may further include positioning an eye cup around the ocular region. The oxygenated material may be dispensed into the eye cup as part of the process of applying the oxygenated material to the ocular region. Some embodiments of the method include coupling a protective shield with the eye cup. In some embodiments, dispensing the oxygenated material into the eye cup includes dispensing the oxygenated material through at a side aperture of the eye cup. In some embodiments, dispensing the oxygenated material into the eye cup includes dispensing the oxygenated material through a transparent layer coupled with a top aperture of the eye cup. Some embodiments include coupling a dispenser with the eye cup to dispense the oxygenated material into the eye cup. In some embodiments, the dispenser may be decoupled from the eye cup; a protective shield may be coupled with the eye cup. Some embodiments include sealing the eye cup around the ocular region. Some embodiments of the method include covering the ocular region after applying the oxygenated material to maintain contact between the ocular region and the oxygenated material.

Applying the oxygenated material to the ocular region may improve healing of the ocular region in some cases. Applying the oxygenated material to the ocular region may facilitate healing of a trauma to the ocular region. Applying the oxygenated material to the ocular region may facilitate preserving tissues in the ocular region. Applying the oxygenated material to the ocular region may occur after a trauma to the ocular region. Applying the oxygenated material to the ocular region may facilitate treatment of at least a disorder of the ocular region, symptoms from the disorder of the ocular region, or a side-effect of a medication.

Some embodiments of the method include identifying at least a trauma to the ocular region or a disorder of the ocular region before applying the oxygenated material to the ocular region. Applying the oxygenated material to the ocular region may at least replace a physiological process of the ocular region or augment the physiological process of the ocular region.

Some embodiments include an ocular region treatment system. The system may include an eye cup configured to surround an ocular region. A dispenser configured to dispense an oxygenated material to the ocular region may be provided. In some embodiments, the dispenser is configured to couple with the eye cup. Some embodiments of the system the dispenser includes aerosol can that may contain the oxygenated material; the aerosol can may be configured to couple with the dispenser.

In some embodiments of the system, at least the dispenser may be configured to decouple from the eye cup or the aerosol can may be configured to decouple from the dispenser. Some embodiments include a protective shield configured to couple with the eye cup to create a closed space to contain the dispensed oxygenated material around the ocular region.

In some embodiments of the system, the oxygenated material includes at least an oxygenated emulsion, an oxygenated ointment, an oxygenated hydrogel, or an oxygenated liquid. The oxygenated liquid, the oxygenated ointment, the oxygenated hydrogel, or the oxygenated emulsion may include at least a supersaturated-oxygenated emulsion, a supersaturated-oxygenated ointment, supersaturated-oxygenated hydrogel, or a supersaturated-oxygenated liquid. The oxygenated material may include perfluorodecalin.

Some embodiments include an ocular region treatment device. The device may include a rigid base with at least a top aperture and a bottom aperture; the bottom aperture may be configured to encompass an ocular region for treatment. The device may include a seal coupled with the rigid base around the bottom aperture. The seal may include a rubber gasket.

Some embodiments of the device include a protective shield configured to couple with and to decouple from the rigid base. Some embodiments of the device include a transparent layer configured to cover the top aperture of the rigid base. The transparent layer may include one or more apertures configured to allow for an oxygenated material to be introduced into the device. The protective shield may include one or more protrusions configured to facilitate the coupling and the decoupling of the protective shield from the rigid base. The protective shield may include one or more apertures configured to facilitate the coupling and the decoupling of the protective shield from the rigid base. The protective shield may include a semi-rigid material. The protective shield may include one or more apertures configured to allow for an oxygenated material to be introduced into the device.

In some embodiments of the device, the rigid base includes one or more side apertures configured to allow for an oxygenated material to be introduced into the device. In some embodiments, the seal includes one or more adhesives to facilitate sealing around the ocular region.

Some embodiments include methods, systems, and/or devices as described in the specification and/or shown in the figures.

The foregoing has outlined rather broadly the features and technical advantages of embodiments according to the disclosure in order that the detailed description that follows may be better understood. Additional features and advantages will be described hereinafter. The conception and specific embodiments disclosed may be readily utilized as a basis for modifying or designing other structures for carrying out the same purposes of the present disclosure. Such equivalent constructions do not depart from the spirit and scope of the appended claims. Features which are believed to be characteristic of the concepts disclosed herein, both as to their organization and method of operation, together with associated advantages will be better understood from the following description when considered in connection with the accompanying figures. Each of the figures is provided for the purpose of illustration and description only, and not as a definition of the limits of the claims.

### BRIEF DESCRIPTION OF THE DRAWINGS

A further understanding of the nature and advantages of different embodiments may be realized by reference to the following drawings. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1A shows a system in accordance with various embodiments.

FIG. 1B shows a system in accordance with various embodiments.

FIG. 2A shows a device in accordance with various embodiments.

FIG. 2B shows a device in accordance with various embodiments.

FIG. 2C shows a device in accordance with various embodiments.

FIG. 2D shows a device in accordance with various embodiments.

FIG. 2E shows a device in accordance with various embodiments.

FIG. 2F shows a device in accordance with various embodiments.

FIG. 2G shows a device in accordance with various embodiments.

FIG. 2H shows a device in accordance with various embodiments.

FIG. 2I shows a device in accordance with various embodiments.

FIG. 2J shows a device in accordance with various embodiments.

FIG. 3A shows a flow diagram of a method in accordance with various embodiments.

FIG. 3B shows a flow diagram of a method in accordance with various embodiments.

FIG. 3C shows a flow diagram of a method in accordance with various embodiments.

FIG. 3D shows a flow diagram of a method in accordance with various embodiments.

### DETAILED DESCRIPTION

This description provides embodiments, and is not intended to limit the scope, applicability or configuration of the disclosure. Rather, the ensuing description will provide those skilled in the art with an enabling description for implementing embodiments of the disclosure. Various changes may be made in the function and arrangement of elements.

Thus, various embodiments may omit, substitute, or add various procedures or components as appropriate. For instance, it should be appreciated that the methods may be performed in an order different than that described, and that various stages may be added, omitted or combined. Also, aspects and elements described with respect to certain embodiments may be combined in various other embodiments. It should also be appreciated that the following systems, devices, and methods may individually or collectively be components of a larger system, wherein other procedures may take precedence over or otherwise modify their application.

Methods, systems, and devices for oxygenated ocular region treatment are provided in accordance with various embodiments. For example, a method of ocular region treatment is provided in accordance with various embodiments where an oxygenated material may be applied to an ocular region. The ocular region may include at least corneal tissue, limbal tissue, ocular adnexal tissue, or skin adjacent to the orbital cavity, for example. The oxygenated material may include at least an oxygenated emulsion, an oxygenated ointment, an oxygenated hydrogel, or an oxygenated liquid, which may be supersaturated with oxygen in some cases. The oxygenated material may include perfluorocarbon, such as perfluorodecalin. The oxygenated material may include at least an antibiotic, anesthetic, or anti-inflammatory in some cases. Some embodiments include an ocular region treatment system that may include an eye cup configured to surround an ocular region. A dispenser configured to couple with the eye cup and to dispense an oxygenated material to the ocular region may be provided.

The tools and techniques provided may have a wide-variety of applications with regard to ocular region treatment. For example, applying the oxygenated material to the ocular region may improve healing of the ocular region in some cases. Applying the oxygenated material to the ocular region may facilitate healing of a trauma to the ocular region. These traumas to the ocular region may occur in both battlefield and non-battlefield situations. The tools and techniques provided may include applying the oxygenated material to the ocular region after a trauma to the ocular region.

The tools and techniques provided may include applying the oxygenated material to the ocular region and may facilitate preserving the ocular region. For example, the application of an oxygenated material (to the ocular region after a trauma, for example) may be utilized in order to promote the preservation of the ocular tissue (i.e. the goal may not be healing). After ocular traumas, care providers may often have an immediate goal that may be to preserve functionality of the tissue, which may allow for treatment options (such as surgeries) to occur at a later date.

In some cases, applying the oxygenated material to the ocular region may facilitate treatment of at least a disorder of the ocular region, symptoms from the disorder of the ocular region, or a side-effect of a medication. For example, the application of an oxygenated material in accordance with various embodiments may be utilized to treat symptoms (from diseases such as glaucoma, for example) as well as side-effects from certain medications. In addition, the application of oxygenated material may be beneficial with respect to conditions like chronic dry eyes, because the eyes may generally utilize tears to promote the exchange of oxygen from the air into the eyes. The application of oxygenated material in accordance with various embodiments may be able to replace or augment a normal physiological process that has been compromised. In some cases, oxygen applied topically to the ocular region may compensate for a temporary imbalance and/or insult to the eye that may disrupt the normal functionality. Some embodiments may supplement normal atmospheric oxygen exchange with a controlled topical oxygenated, ointment, liquid, or emulsion based oxygen exchange.

Some embodiments include treatment that involves application of a liquid perfluorodecalin (PFD)/O₂ emulsion to the eye. The therapeutic benefit may include at least an increase in re-generation of damaged sclera, conjunctiva, cornea, or ocular adnexa, or acceleration of ocular wound healing, for example. Some embodiments may leverage the regenerative properties and wound healing characteristics of PFD/O₂ to develop a topical therapeutic for various types of ocular trauma or other ocular related issues.

During wound healing, cellular consumption of oxygen increases due to higher metabolic demand. This is also true of ocular tissues, and healing in the eye is therefore dependent upon sufficient oxygenation. The cornea acquires this oxygen through the tears, which absorb and deliver oxygen from the atmosphere. Some topical therapies can therefore deprive the eye of oxygen and inhibit ocular healing.

The concept of delivering high levels of topical oxygen to the eye, perhaps as a PFD/O₂ emulsion, may have previously been avoided or overlooked due to the idea that high levels of oxygen may cause damage to ocular tissues. This idea may stem from a limited number of studies that indicate that hyperbaric oxygen therapy may lead to acute and reversible changes in visual acuity, but only after hours of exposure and only in a small subset of individuals. It may be the case that high levels of topical oxygen may promote wound healing, including healing in epithelial tissues. The outer portion of the cornea is generally composed of epithelial tissue. Thus, the topical application of a high concentration of O₂ to the eye, or more generally the ocular region, may promote healing, such as wound healing. In some cases, there may be a threefold to fivefold increase in corneal interstitial pO₂ levels, though other increases may be seen in some embodiments. There may be significant acceleration of corneal and/or ocular adnexal wound healing.

Some embodiments involve specific treatment protocols. For example, there may be the application of PFD/O₂ cream or liquid emulsion to the eye in concert with post-treatment application of a patch. Some embodiments may include treatments and/or applicators designed for self-use and/or field use by medics. Some embodiments may be configured for hospital or clinical applications. Embodiments configured for hospital or clinical applications may include use for the preservation of donor and/or recipient ocular tissues during transplantation (i.e. cornea transplantation). In some cases, use of various embodiments for tissue preservation during cornea transplantation may prevent neovascularization of the transplanted cornea.

There may be different therapeutic benefits from the variety of embodiments. For example, PFD/O₂ emulsion may trigger a significant up-regulation of key substances involved in the various stages of wound healing, such as Type I collagen. One may note that Type I collagen may be critical to regeneration and/or healing of cornea, sclera, conjunctiva and/or adnexal tissue. Some embodiments may improve healing of ocular adnexal (i.e. eye lid) injuries and injuries to the skin adjacent to the orbital cavity.

In some cases of ocular injury, intraocular pressure can become elevated and cause further damage to ocular tissues, including the retina. Some embodiments, such as a PFD/O₂ emulsion, may decrease intraocular pressure, which may be beneficial in cases of ocular injuries where intraocular pressure has become elevated. Some embodiments may decrease intraocular pressure by causing vasoconstriction due to enhanced tissue oxygenation.

Some embodiments may involve reformulating PFD/O₂ emulsions used for skin wound healing for ocular applications. Some embodiments may include an eye applicator and/or treatment protocol that may allow for easy self-use and/or field use, though other embodiments may include hospital and/or clinic-specific protocols and/or applicators.

Some embodiments may help address a growing problem of ocular injury in battlefield settings. There may be a widening gap in treatment, which may be due to the continued use of antiquated protocols and a dearth of therapeutic agents. For example, there may still be widespread continued use of outdated “Eye Trauma” first aid kits that may promote the incorrect treatment of ocular injuries using absorbent eye patches. Unlike other battlefield injuries involving significant bleeding in which direct pressure may be most effective, proper pre-hospital care for eye injury may generally include, first and foremost, preventing pressure to the eye, which may prevent expulsion of intraocular contents by placing a rigid shield over the eye during transport to definitive ophthalmic care.

Some embodiments may address the problem that there may be few non-surgical treatments for severe ocular trauma, and/or post-surgical treatment of wounds to promote ocular wound healing. Topical treatment typically may be limited to the application of an antibiotic to avoid infection and/or anesthetic/analgesic to reduce sensitivity and pain. Penetrating wounds may generally demand surgical repair, whereas many forms of non-penetrating ocular trauma may often be treated with topical antibiotic and anesthetic/analgesic agents alone.

For example, corneal abrasion may often be caused by contact with a foreign object and may result in an extremely painful eye that may involve topical anesthetic/analgesic application prior to treatment. Treatment also typically may involve an antibiotic to avoid infection. Simple partial-thickness (lamellar) lacerations may often be treated without suturing, whereas deeper lacerations may involve suturing. Regardless of the use of suturing, most lacerations may generally be cleaned and treated with systemic antibiotics. Burns may affect the ocular adnexal tissues (i.e., eyelids and/or conjunctiva) in addition to cornea, and proper treatment protocols may demand the affected areas remain moist and free from exposure. Non-surgical treatment of ocular burns typically may be limited to applying antibiotic ointment generously all over the conjunctiva, cornea and burned eyelids.

In general, treatment protocols for non-penetrating ocular trauma may focus on reducing infection while managing sensitivity and pain. Other than surgical repair, few treatment protocols may exist to accelerate the rate of tissue growth and healing.

The methods, systems, and devices provided in accordance with various embodiments may help address one or more of these issues. For example, some embodiments may involve the topical application of a liquid perfluorocarbon/oxygen emulsion (some embodiments utilize perfluorodecalin or PFD/O₂) directly to the eye. The therapeutic benefits may include an attenuation of pain, a significant up-regulation of the expression of Type I collagen, prevention of neovascularization, a decrease in intraocular pressure, a correlated increase in the re-generation of damaged sclera, conjunctiva, and/or epithelial corneal layers as well as ocular adnexal dermal layers, and/or a resulting acceleration in the rate of ocular wound healing.

In some embodiments, supersaturated-oxygen-containing emulsion, or oxygenated emulsions in general, may be designed to slowly release its entrapped oxygen over time. The oxygen solubility of PFD may be relatively high (approximately 20 times greater than water, for example); therefore, it may have a high oxygen-carrying capacity. During manufacture, oxygen may be dissolved into the PFD emulsion and may be stored under pressure in a small can. By maintaining pressure on the PFD/O₂ emulsion, dissolution and out gassing may be prevented during storage and a maximum oxygen concentration may be delivered on dispensation in some cases. The topical solution may be formulated with biocompatible emulsifying agents, which may ensure adequate stability of the dispersed PFD/O₂ emulsion. Before dispensation, the dissolved oxygen concentration contained in the topical solution may be approximately 2.0 mL of oxygen (standard temperature and pressure) per milliliter of PFD in some embodiments; other embodiments may utilize other oxygen concentrations. After dispensation, this combined PFD/O₂ emulsion may cause a local increase of 3-5 times greater partial pressure of oxygen in some cases, for example.

A variety of oxygenated PFD formulations may be utilized. Merely by way of example, a 30% PFD by volume saturated with O₂ may be utilized in some embodiments; other embodiments may utilize a 55% PFD by volume saturated by O₂ may be utilized. Other percentages of PFD may also be utilized in some embodiments. These examples of 30% PFD by volume and 55% PFD by volume may have specific gravities slightly higher than 1.0. This may be more in line with the specific gravity of aqueous or subretinal fluids.

In vitro studies may suggest that the application of oxygenated materials such as PFD/O₂ emulsions may enhance cell viability in a dose-dependent fashion. The enhancement of viability above control levels may imply that the use of oxygenated materials may not only help preserve cell viability, but also may promote cellular proliferation. This may have significant implications for ocular region healing, as proliferation may be a key step in the healing process. Furthermore, in vitro studies suggest that the use of oxygenated materials may promote cell survival in the critical and delicate limbal cell population following injury. In vitro studies also suggest that delivering more oxygen through the use of oxygenated materials to ocular region cells may be beneficial and may promote improvements for healing, such as wound healing. Furthermore, in vitro studies suggest that application of oxygenated materials such so PFD/O₂ do not promote apoptosis or DNA damage.

Some embodiments may be configured to eliminate eye sensitivity concerns while providing proper flow and/or wetting for uniform coverage upon application.

Oxygenated perfluorocarbon emulsions that may have high stability and good oxygen release have generally been used as artificial oxygen carriers in other medical applications. These emulsions may typically include a disperse phase of an oxygenated perfluorocarbon in an aqueous solution. Most oxygenated perfluorocarbons are generally highly polar, which may create a natural phase separation from the aqueous solution and may promote uniform dispersion of the emulsion in the solution. Emulsions of this type may be prepared in either liquid or hydrogel solutions depending on the desired viscosity, flow, and /or drying characteristics for the particular application.

Perfluorocarbon liquids like PFD generally may be considered to be “eye safe” and some may be frequently used as intraoperative tools in vitreoretinal surgery. While perfluorocarbon liquids may have high specific gravity relative to aqueous or subretinal fluid, which may practically limit their use in intra-ocular procedures to temporary applications such as retinal tamponades, there may be no known reasons for restricting their use in topical applications to the eye. Some embodiments may include PFD/O₂ emulsion formulation that may be assessed for eye safety and eye sensitivity, and may be formulated developed using acceptable (and perhaps FDA-approved and commercially available) PFD liquids, anesthetic/analgesic agents and other eye-safe additives to yield flow and wetting properties that may be compatible with application to the eye surface. Some embodiments may utilize PFD/O₂ emulsion technology for the treatment of ocular trauma or other conditions configured with respect to viscosity, flow, and/or drying characteristics to be more compatible with ocular tissues.

Some embodiments include an applicator design that may allow for easy self-application and/or application by trained medics. In some embodiments, the applicator may have several main components: 1) a pressurized aerosol cartridge of the PFD/O₂ emulsion (similar to a shaving gel cartridge); 2) a dispenser into which the aerosol cartridge may be attached and that may provide nozzles to properly distribute the emulsion; 3) a hand-operated lever (or levers) that may control the flow of emulsion, and/or 4) a comfortable eye cup that may seal around the orbit of the eye to contain the emulsion and protect the eye pre- and post-treatment. The applicator may be designed for easy use, and special consideration may be given to features that may minimize discomfort and may promote effective and uniform distribution of the emulsion to the eye and ocular adnexal areas. In some embodiments, the eye cup may be easily replaced by a rigid eye patch after treatment, or it may be designed to function as a rigid eye patch by the addition of a cover. In some embodiments, the eye cup may be manufactured using appropriate surgical-grade materials and engineered with features to promote proper ventilation.

Some embodiments may include a treatment protocol that may involve regular (e.g., twice daily) application of the emulsion to the injured tissue using the applicator for some prescribed period depending on the severity of the wounds (days or weeks, as necessary). The applicator eye cup or other protective eye patch may be replaced every few days to cleanse the wound and assess the healing process, for example. The emulsion may also be applied to the ocular region without the accompaniment of an eye cup or protective eye patch in some cases.

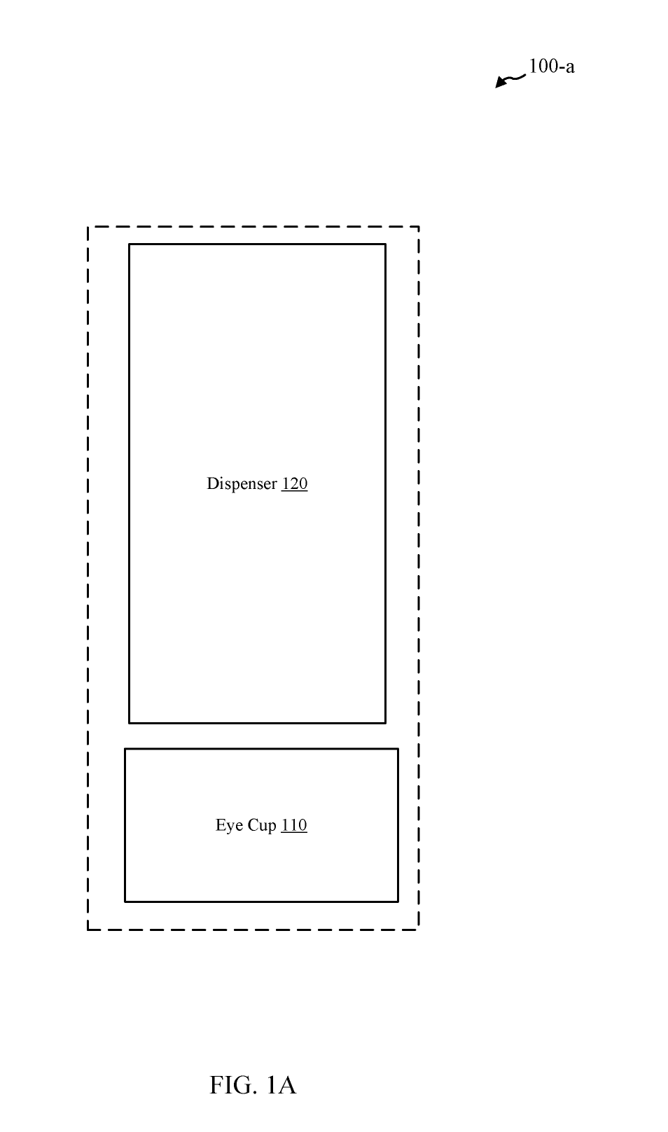

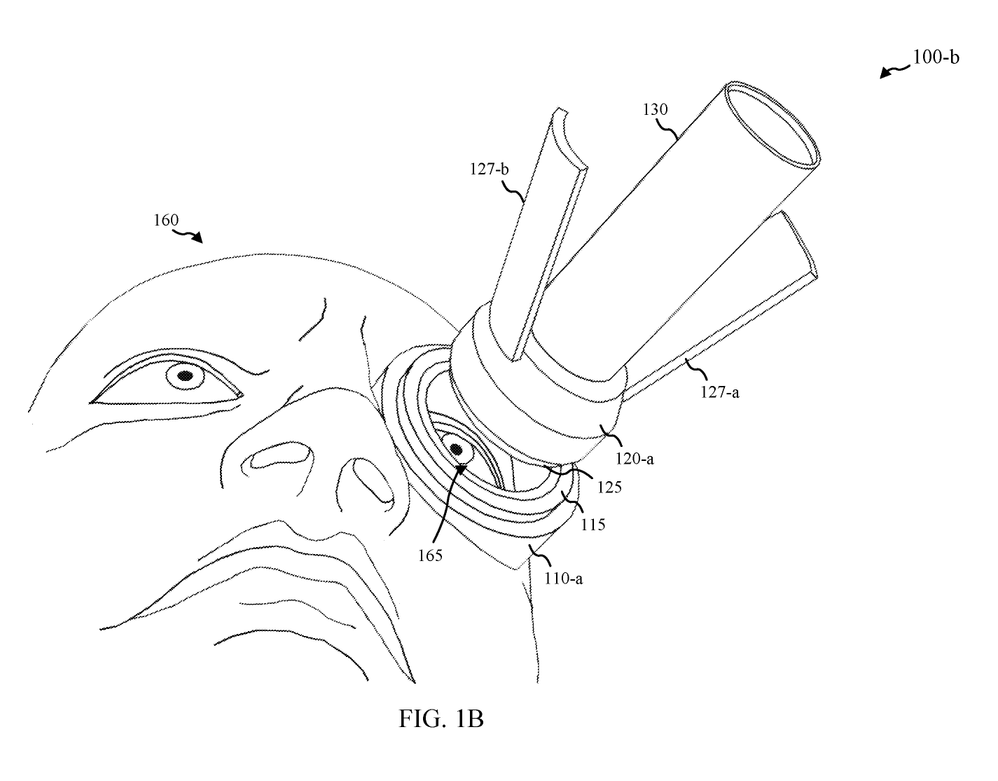

Turning now to FIG. 1A, an ocular region treatment system 100-a is provided in accordance with various embodiments. System 100-a may include a dispenser 120 and/or an eye cup 110. The eye cup 110 may be configured to surround an ocular region. The dispenser 120 may be configured to dispense an oxygenated material to the ocular region. The dispenser 120 may be configured to couple with the eye cup 110. Some embodiments of the system 100-a include an aerosol can (not shown, see FIG. 1B, for example) containing the oxygenated material, where the aerosol can may be configured to couple with (or be part of) the dispenser 120.

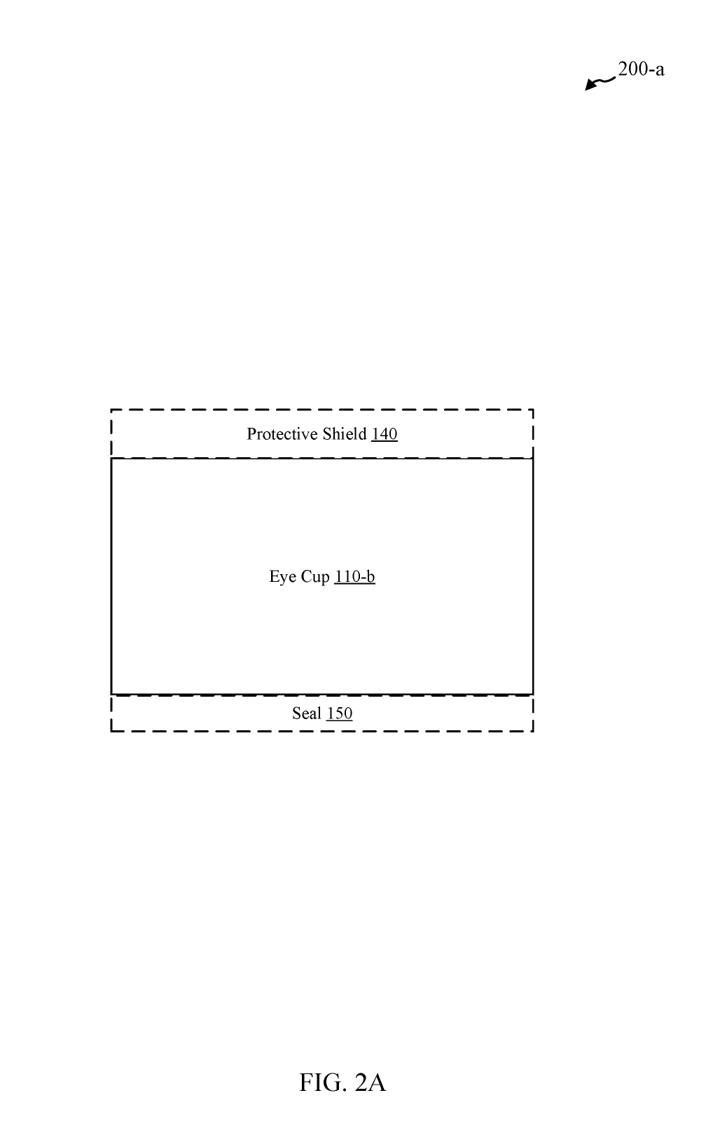

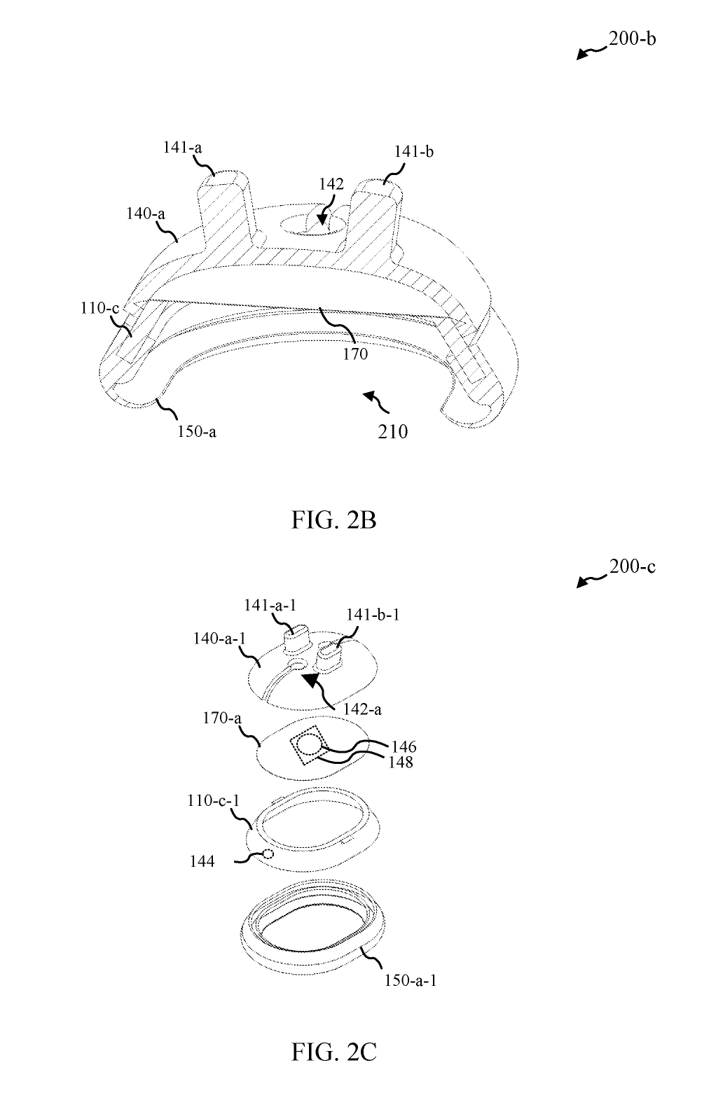

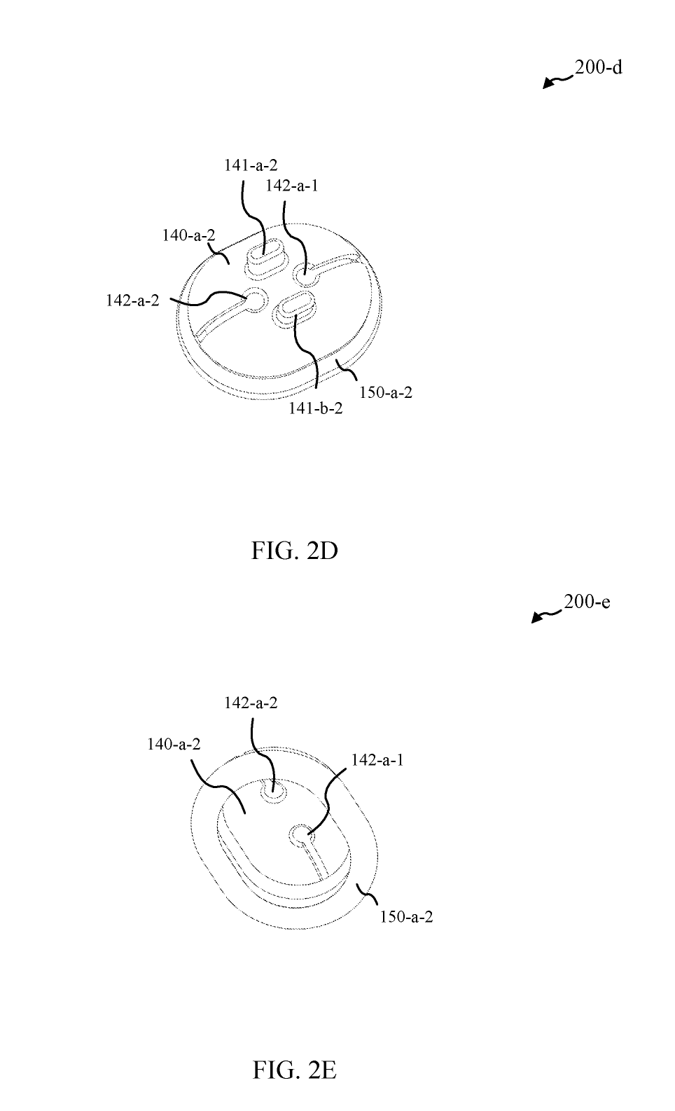

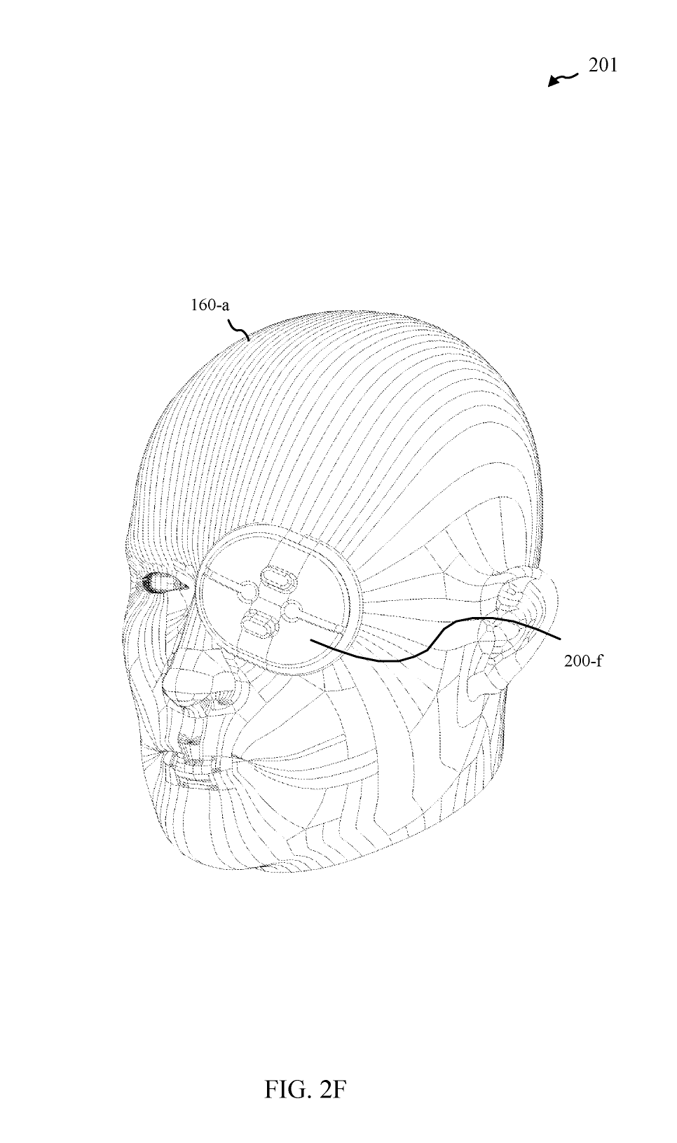

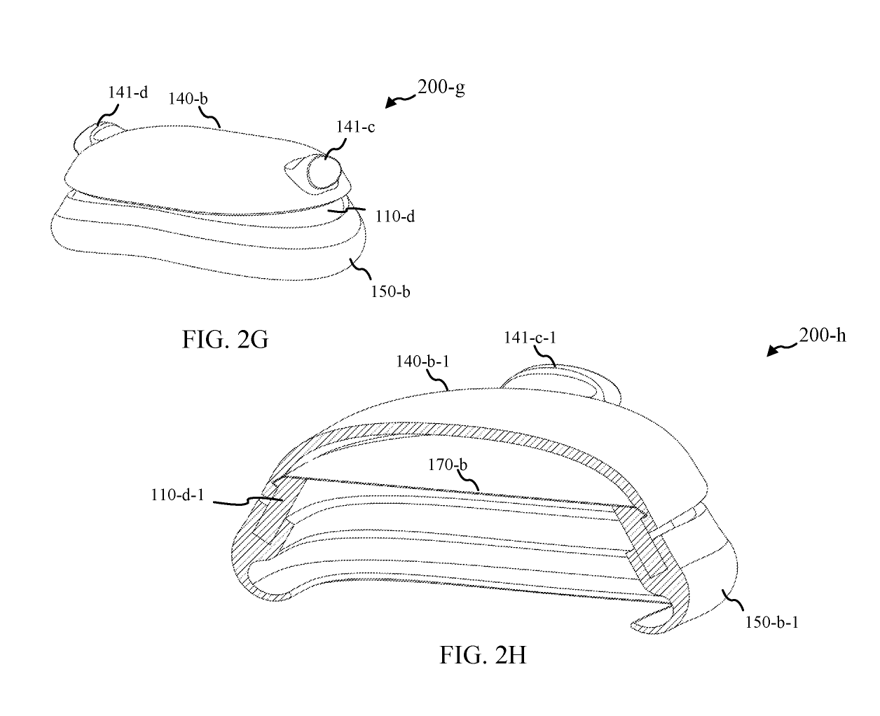

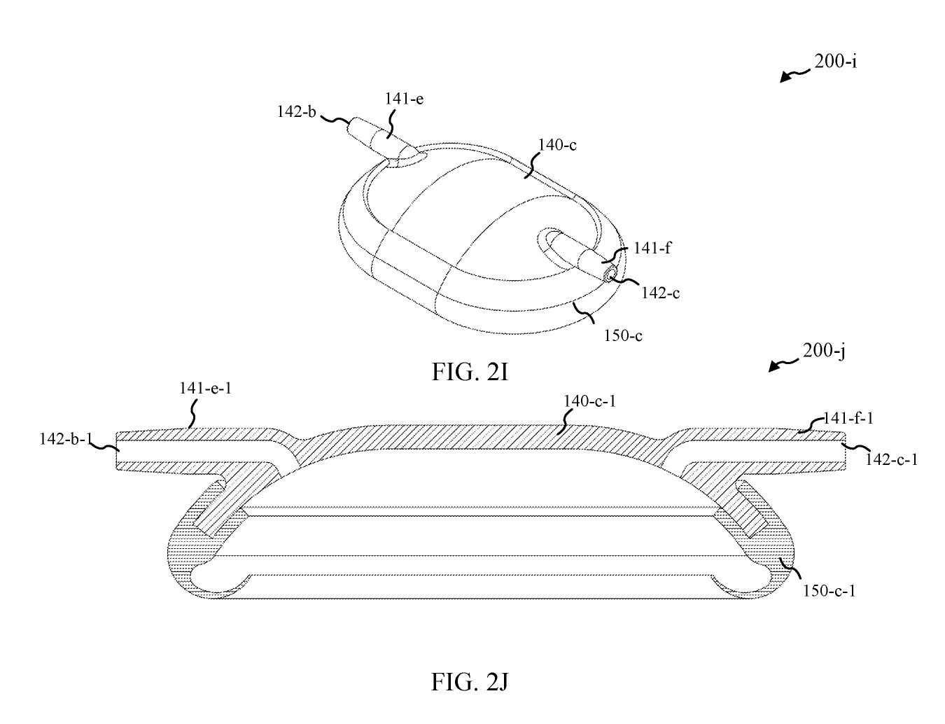

In some embodiments, at least the dispenser 120 may be configured to decouple from the eye cup 110 or the aerosol can may be configured to decouple from the dispenser 120. Some embodiments include a protective shield (not shown, see FIG. 2A, for example) configured to couple with the eye cup 110 to create a closed space to contain the dispensed oxygenated material around the ocular region.

In some embodiments of the system 100-a, the oxygenated material includes at least an oxygenated emulsion, an oxygenated ointment, an oxygenated hydrogel, or an oxygenated liquid. The oxygenated liquid, the oxygenated ointment, the oxygenated hydrogel, or the oxygenated emulsion may include at least a supersaturated-oxygenated emulsion, a supersaturated-oxygenated ointment, a supersaturated-oxygenated hydrogel, or a supersaturated-oxygenated liquid. The oxygenated material may include perfluorodecalin.

FIG. 1B shows an example of an ocular region treatment system 100-b in accordance with various embodiments. System 100-b may be an example of system 100-a of FIG. 1A, for example. System 100-b may include a dispenser 120-a and/or an eye cup 110-a.

In this example, a head region 160 of a patient is shown along with an ocular region 165. The eye cup 110-a may be configured to surround the ocular region 165. The dispenser 120-a may be configured to couple with the eye cup 110-a, utilizing one or more interface rings 125 and/or 115. The dispenser 120-a may dispense an oxygenated material to the ocular region 165. System 100-b also includes a container 130, such as an aerosol can, that may contain the oxygenated material, where the container 130 may be configured to couple with (or be part of) the dispenser 120-a. Dispenser 120-a may also include components such as hand-operated levers 127-a/127-b that may be utilized to help dispense the oxygenated material from the container 130.

Dispenser 120-a may be configured to decouple from the eye cup 110-a or the aerosol can 130 is configured to decouple from the dispenser 120-a. Some embodiments include a protective shield (see FIG. 2A, for example) configured to couple with the eye cup 110-a to create a closed space to contain the dispensed oxygenated material around the ocular region.

In some embodiments of the system 100-b, the oxygenated material includes at least an oxygenated emulsion, an oxygenated ointment, an oxygenated hydrogel, or an oxygenated liquid. The oxygenated liquid, the oxygenated ointment, the oxygenated hydrogel, or the oxygenated emulsion may include at least a supersaturated-oxygenated emulsion, a supersaturated-oxygenated ointment, a supersaturated-oxygenated hydrogel, or a supersaturated-oxygenated liquid. The oxygenated material may include perfluorodecalin.

Turning now to FIG. 2A, an ocular region treatment device 200-a in accordance with various embodiments is provided. Device 200-a may include an eye cup 110-b, which may be an example of eye cup 110 of FIG. 1A and/or eye cup 110-a of FIG. 1B. Device 200-a may be utilized as an aspect of systems 100-a of FIG. 1A and/or system 100-b of FIG. 1B.

Device 200-a may include a protective shield 140 that may be configured to couple with the eye cup 110-b, which may create a closed space to contain dispensed oxygenated material around the ocular region. Device 200-a may include a seal 150 that may facilitate sealing the eye cup 110-b around an ocular region. The eye cup 110-b may include a rigid base with at least a top aperture and a bottom aperture; the bottom aperture may be configured to encompass an ocular region for treatment. The seal 150 may be coupled with the rigid base of eye cup 110-b around the bottom aperture. The seal may include a rubber gasket.

In some embodiments, the protective shield 140 is configured to couple with and to decouple from the rigid base of eye cup 110-b. Some embodiments of the device 200-a include a transparent layer configured to cover the top aperture of the rigid base of eye cup 110-b; in some embodiments, the transparent layer may be considered as an aspect of the protective shield 140. The transparent layer may include one or more apertures configured to allow for an oxygenated material to be introduced into the device 200-a. The protective shield 140 may include one or more protrusions configured to facilitate the coupling and the decoupling of the protective shield 140 from the rigid base of the eye cup 110-b. Other techniques may be utilized to couple the protective shield 140 with the rigid base of the eye cup 110-b; for example, portions of the protective shield 140 and the rigid base of the eye cup 110-b may be threaded such that they two components may be screwed together. In some embodiments, an adhesive may be utilized to couple these two components with each other. The protective shield 140 may include one or more apertures configured to facilitate the coupling and the decoupling of the protective shield 140 from the rigid base of the eye cup 110-b. The protective shield 140 may include a semi-rigid material. The protective shield 140 may include one or more apertures configured to allow for an oxygenated material to be introduced into the device 200-a.

In some embodiments of the device 200-a, the rigid base of the eye cup 110-b includes one or more side apertures configured to allow for an oxygenated material to be introduced into the device 200-a. In some embodiments, the seal 150 includes one or more adhesives to facilitate sealing around the ocular region.

Turning now to FIG. 2B, FIG. 2C, FIG. 2D, and FIG. 2E, different perspectives 200-b, 200-c, 200-d, and 200-e of an ocular region treatment device in accordance with various embodiments are provided. This ocular region treatment device may be an example of 200-a of FIG. 2A, for example.

Perspective 200-b of the ocular region treatment device may show a cross-sectional view of the device, where the device may include a protective shield 140-a, a rigid base 110-c, and/or a seal 150-a. The protective shield 140-a may include one or more protrusions 141-a/141-b that may be configured to facilitate the coupling and the decoupling of the protective shield 140-a from the rigid base 110-c. For example, protrusions 141-a/141-b may be pinched towards each other to allow the protective shield 140-a to couple with the rigid base 110-c; this technique may also be utilized to decouple the protective shield 140-a from the rigid base 110-c. The protective shield 140-a may include one or more apertures 142 that may be configured to facilitate the coupling and the decoupling of the protective shield 140-a from the rigid base 110-c. In some embodiments, the one or more apertures 142 may be configured to allow for an oxygenated material to be introduced into the device 200-b. Some embodiments include a transparent layer 170 configured to cover the top aperture of the rigid base 110-c; in some embodiments, the transparent layer may be considered as an aspect of the protective shield 140-a. The perspective of device 200-b may show an interior chamber 210 that may be formed by the device.

Perspective 200-c of the ocular region treatment device may show an exploded view of the device, where the device may include a protective shield 140-a-1, a rigid base 110-c-1, and/or a seal 150-a-1. The protective shield 140-a-1 may include one or more protrusions 141-a-1/141-b-1 that may be configured to facilitate the coupling and the decoupling of the protective shield 140-a-1 from the rigid base 110-c-1. The protective shield 140-a-1 may include one or more apertures 142-a that may be configured to facilitate the coupling and the decoupling of the protective shield 140-a-1 from the rigid base 110-c-1. In some embodiments, the one or more apertures 142-a may be configured to allow for an oxygenated material to be introduced into the device 200-c. Some embodiments may include an aperture and/or channel 144 formed in the base 110-c-1 to allow for the oxygenated material to be introduced into the device 200-c.

Perspective 200-c may also show a transparent layer 170-a. The transparent layer 170-a may be configured to cover the top aperture of the rigid base of eye cup 110-c-1; in some embodiments, the transparent layer 170-a may be considered as an aspect of the protective shield 140-a-1. The transparent layer 170-a may include one or more apertures 146 that may be configured to allow for an oxygenated material to be introduced into the device 200-c. In some embodiments, a moveable flap 148 may be coupled with the transparent layer 170-a, which may be utilized to cover the aperture 146, but may be moveable in order to allow for the introduction of the oxygenated material into the device 200-c.

FIG. 2D and FIG. 2E provide a top perspective 200d and a bottom perspective 200-e of the ocular region treatment device in accordance with various embodiments. These two perspectives may show, in particular, seal 150-a-2, protective shield, 140-a-2, and/or apertures 142-a-1/142-a-2. Perspective 200d may also show protrusions 141-a-2/141-b-2.

Turning now to FIG. 2F, a perspective 201 that may involve utilizing an ocular region treatment device 200-f in accordance with various embodiments is provided. Device 200-f may be an example of the ocular region treatment devices of FIG. 2A, FIG. 2B, FIG. 2C, FIG. 2D, and/or FIG. 2E. In this example, a head region 160-a of a patient is shown along with an ocular region treatment device 200-f in place over an ocular region to be treated with oxygenated material. The device 200-f may be configured to surround the ocular region. In some embodiments, the device 200-f may be secured in place utilizing a variety of techniques. For example, an adhesive may be utilized to hold the device 200-f in place; the adhesive may be part of a seal. Other techniques may be utilized such utilizing an external wrap around the head region 160-a and the device 200-f may be utilized to hold the device 200-f in place.

Turning now to FIG. 2G and FIG. 2H,a side perspective 200-g of an ocular region treatment device and a cross-sectional view 200-h of an ocular region treatment device are provided in accordance with various embodiments. These may be examples of low-profile ocular region treatment devices. Device 200-g may include, for example, a protective shield 140-b that may include protrusions 141-c/141-d. Device 200-g may also show rigid base 110d along with seal 150-b. Similarly, device 200-h may show, for example, a protective shield 140-b-1 that may include protrusions 141-c-1. Device 200-h may also show rigid base 110-d-1 along with seal 150-b-1. Device 200-h may also show transparent layer 170-b.

Turning now to FIG. 2I and FIG. 2J, a side perspective 200-i of an ocular region treatment device and a cross-sectional view 200-j of an ocular region treatment device are provided in accordance with various embodiments. These may be examples of additional low-profile ocular region treatment devices. Device 200-h may include, for example, a protective shield 140-c that may include protrusions 141-e/141-f. Device 200-h may a seal 150-c. In some embodiment, the protective shield 140-c may be considered part of a rigid base of the eye cup. The protrusions 141-e/141-f may include apertures and/or channels 142-b/142-c that may allow for the introduction of oxygenated material to a chamber formed by the device 200-i. Similarly, device 200-j may show, for example, a protective shield 140-c-1 that may include protrusions 141-e-1/141-f-1. Device 200-h may a seal 150-c-1. In some embodiment, the protective shield 140-c-1 may be considered part of a rigid base of the eye cup. The protrusions 141-e-1/141-f-1 may include apertures and/or channels 142-b-1/142-c-1 that may allow for the introduction of oxygenated material to a chamber formed by the device 200-j.

The ocular region treatment devices 200 of FIG. 2A, FIG. 2B, FIG. 2C, FIG. 2D, FIG. 2E, FIG. 2F, FIG. 2G, FIG. 2I, and/or FIG. 2J in accordance with various embodiments provide a hybrid eye cup and/or wound applicator design. In general, these may be utilized to facilitate application of an oxygenated material (such as a supersaturated-oxygenated emulsion, for example) as a topical therapeutic and may be applicable for a broad range of ocular region treatments. In some embodiments, the seals 150 may be made of a soft rubber gasket and may conform easily to, and may seal against, the structure surrounding the ocular region (e.g., cheeks, nose, forehead, etc.). The rigid bases 110 may support the seals 150. And while the bases 110 may in general be rigid, in some embodiments, the bases 110 may be semi-rigid. The bases 110 may be referred as an eye cup in some embodiments. In other embodiments, the combination of one or more of seal 150, base 110, and/or protective shield 140 may be referred to as an eye cup. Some embodiments may include the transparent layer 170, which may in general include a clear plastic film that may cover the base 110 and maintain the oxygenated material within the interior chamber formed by the ocular region treatment device when positioned with respect to the ocular region for treatment. The protective shields 140 may be formed from a semi-rigid to rigid material that may be coupled and/or decoupled from the base 110 and/or seal 150 in different embodiments. Some embodiments may utilize the protrusions 141 in general to facilitate the coupling and/or decoupling.

In some embodiments, the protective shield 140 may serve to block out light, such as sunlight, while also providing protection to the ocular region. When the protective shield may be removed, it may expose the transparent layer 170 that may seal the interior chamber portion of the assembly. The oxygenated material or other liquid, cream, emulsion, or gel topical therapies may be administered either through an opening in the transparent layer 170 or through an aperture or channel in the base 110 or the protective shield 140.

In general, system 100-a of FIG. 1A, system 100-b of FIG. 1B, and/or devices 200 of FIG. 2A, FIG. 2B, FIG. 2C, FIG. 2D, FIG. 2E, FIG. 2F, FIG. 2G, FIG. 2H, FIG. 2I, and/or FIG. 2J may be utilized with regard to numerous methods for ocular region treatment where an oxygenated material may be applied to an ocular region. In some cases, the ocular region may include at least corneal tissue or limbal tissue. In some cases, the ocular region includes ocular adnexal tissue.

With respect to the use of system 100-a of FIG. 1A, system 100-b of FIG. 1B, and/or devices 200 of FIG. 2A, FIG. 2B, FIG. 2C, FIG. 2D, FIG. 2E, FIG. 2F, FIG. 2G, FIG. 2H, FIG. 2I, and/or FIG. 2J, the oxygenated material may include at least an oxygenated emulsion, an oxygenated ointment, or an oxygenated liquid. The oxygenated emulsion, the oxygenated ointment, or the oxygenated liquid may include at least a supersaturated-oxygenated emulsion, a supersaturated-oxygenated ointment, or a supersaturated-oxygenated liquid. In some embodiments, the oxygenated material includes perfluorocarbon. The perfluorocarbon may include perfluorodecalin. The oxygenated material may include at least an antibiotic or an anesthetic in some cases. In some embodiments, the oxygenated material is configured to produce a partial pressure of O₂ above ambient atmospheric pressure when applied to the ocular region.

The use of system 100-a of FIG. 1A, system 100-b of FIG. 1B, and/or devices 200 of FIG. 2A, FIG. 2B, FIG. 2C, FIG. 2D, FIG. 2E, FIG. 2F, FIG. 2G, FIG. 2H, FIG. 2I, and/or FIG. 2J may include positioning an eye cup 110 around the ocular region. A dispenser, such as dispenser 120 of FIG. 1A and/or 120-a of FIG. 1B, may be coupled with the eye cup 110. The oxygenated material may be dispensed into the eye cup 110 as part of the process of applying the oxygenated material to the ocular region. In some embodiments, the dispenser 120 may be decoupled from the eye cup 110; protective shield, such as protective shield 140 of FIG. 2A, FIG. 2B, FIG. 2C, FIG. 2D, FIG. 2E, FIG. 2F, FIG. 2G, FIG. 2H, FIG. 2I, and/or FIG. 2J, may be coupled with the eye cup 110. Some embodiments include sealing the eye cup 110 around the ocular region, such as with a seal, like seal 150 of FIG. 2A, FIG. 2B, FIG. 2C, FIG. 2D, FIG. 2E, FIG. 2F, FIG. 2G, FIG. 2H, FIG. 2I, and/or FIG. 2J.

The use of system 100-a of FIG. 1A, system 100-b of FIG. 1B, and/or devices 200 of FIG. 2A, FIG. 2B, FIG. 2C, FIG. 2D, FIG. 2E, FIG. 2F, FIG. 2G, FIG. 2H, FIG. 2I, and/or FIG. 2J may include covering the ocular region after applying the oxygenated material to maintain contact between the ocular region and the oxygenated material. Applying the oxygenated material to the ocular region may improve healing of the ocular region in some cases. Applying the oxygenated material to the ocular region may facilitate healing of a trauma to the ocular region. Applying the oxygenated material to the ocular region may facilitate preserving the ocular region. Applying the oxygenated material to the ocular region may occur after a trauma to the ocular region. Applying the oxygenated material to the ocular region may facilitate treatment of at least a disorder of the ocular region, symptoms from the disorder of the ocular region, or a side-effect of a medication.

In some cases, the application of an oxygenated material (to the ocular region after a trauma, for example) may be utilized in order to promote the preservation of the ocular tissue (i.e. the goal may not be healing). After ocular traumas, care providers may often have an immediate goal that may be to preserve functionality of the tissue, which may allow for treatment options (such as surgeries) to occur at a later date.

In some cases, the application of an oxygenated material in accordance with various embodiments may be utilized to treat symptoms (from diseases such as glaucoma, for example) as well as side-effects from certain medications. In addition, the application of oxygenated material may be beneficial with respect to conditions like chronic dry eyes, because the eyes may generally utilize tears to promote the exchange of oxygen from the air into the eyes. The application of oxygenated material in accordance with various embodiments may be able to replace or augment a normal physiological process that has been compromised. In some cases, oxygen applied topically to the ocular region may compensate for a temporary imbalance and/or insult to the eye that may disrupt the normal functionality. Some embodiments may supplement normal atmospheric oxygen exchange with a controlled topical oxygenated, ointment, liquid, or emulsion based oxygen exchange.

The use of system 100-a of FIG. 1A, system 100-b of FIG. 1B, and/or devices 200 of FIG. 2A, FIG. 2B, FIG. 2C, FIG. 2D, FIG. 2E, FIG. 2F, FIG. 2G, FIG. 2H, FIG. 2I, and/or FIG. 2J may include identifying at least the trauma to the ocular region or the disorder of the ocular region before applying the oxygenated material to the ocular region. Applying the oxygenated material to the ocular region may replace or augment a physiological process of the ocular region.

System 100-a of FIG. 1A, system 100-b of FIG. 1B, and/or devices 200 of FIG. 2A, FIG. 2B, FIG. 2C, FIG. 2D, FIG. 2E, FIG. 2F, FIG. 2G, FIG. 2H, FIG. 2I, and/or FIG. 2J may be utilized to provide supplement corneal oxygen supply, which may promote re-epithelialization. For example, without blood vessels, the cornea generally gets oxygen directly from the air. In a healthy cornea, atmospheric oxygen may first dissolve in the tears and may then diffuse throughout the cornea. In an injured cornea, epithelial cells of conjunctival origin may cover the exposed corneal surface in order to initiate healing. Assuming adequate oxygenation, four to five weeks after re-epithelialization, these cells may undergo a morphologic transformation to normal-appearing corneal epithelium. While the specific roles that oxygen plays in promoting wound healing may still not be well established, the range of possible mechanisms may include: degradation of necrotic wound tissue, potential up-regulation of key human growth factors (EGF, HGF, TGF-b, and PDGF-BB), triggering expression of other immunoproteins, such as CAP37, and/or stimulation of neutrophil-mediated oxidative microbial killing.

Some embodiments may utilize system 100-a of FIG. 1A, system 100-b of FIG. 1B, and/or devices 200 of FIG. 2A, FIG. 2B, FIG. 2C, FIG. 2D, FIG. 2E, FIG. 2F, FIG. 2G, FIG. 2H, FIG. 2I, and/or FIG. 2J to provide topical therapy that may supplement corneal oxygen supply to better promote re-epithelialization and thereby may provide a compliment to other topical and surgical clinical treatments that otherwise may starve the corneal epithelium of necessary oxygen.

Systems and/or devices as shown in system 100-a of FIG. 1A, system 100-b of FIG. 1B, and/or devices 200 of FIG. 2A, FIG. 2B, FIG. 2C, FIG. 2D, FIG. 2E, FIG. 2F, FIG. 2G, FIG. 2H, FIG. 2I, and/or FIG. 2J may be utilized to supplement impaired limbal tissue perfusion, which may avoid delayed limbal stem cell deficiency. Regardless of the trauma or post-traumatic clinical treatment, re-epithelialization may general involve a viable limbal stem cell population. Delayed Limbal Stem Cell Deficiency (LSCD) and/or resulting non-recoverable vision loss may occur from combat-induced ocular trauma. In some cases, there may be the presence of a 3-4 day post-trauma therapeutic window during which intervention may help avoid delayed LSCD. LSCD may result from chemical or thermal burns, microbial infections, sulfur mustard gas poisoning, and/or other traumatic assaults that may interrupt O₂ perfusion to the limbus and may compromise the limbal stem cell population.

In general, oxygen may be supplied to the cornea through surface absorption from the air via the thin layer of tear fluid covering the cornea. Conversely, oxygen levels in the anterior chamber angle (including the limbus) may be strongly influenced by oxygen from the ciliary body circulation. Many types of battlefield ocular trauma, for example, may impair limbal tissue perfusion through the ciliary vasculature, and may promote delayed LSCD.

Some embodiments may supplement impaired limbal tissue perfusion for a sufficient time so as to prevent or delay the onset of LSCD and may preserve as many clinical treatment options as possible for the casualty.

In the case of non-penetrating ocular trauma care, it may be desirable to utilize a supersaturated oxygenated emulsion (SOE) topical product that may be compatible with Echelon I and/or Echelon II medic-applied antibiotic ointments and that promotes more rapid re-epithelialization as well as arrests the development of delayed LSCD.

In the case of suspected penetrating ocular trauma care, it may be desirable to develop an SOE topical product that is compatible with Echelon I medic-applied SHIELD-AND-SHIP protocols and devices (e.g., rigid eye shields) and supplements impaired limbal tissue perfusion (and onset of delayed LSCD) until surgical treatment may be practical.

Casualties evacuated to Echelon III care or other clinical care facilities may generally be for ocular trauma usually involving surgical procedures. For this clinical point of care, it may be desirable to develop an SOE topical product that is compatible with current prophylaxis (e.g., corneal band-aids, etc.) and supplements both corneal oxygen supply to promote re-epithelialization and impaired limbal tissue perfusion to prevent delayed LSCD.

Some methods, system, and devices provided may be applicable in the event of trauma to the ocular region in which the blood flow through the ciliary vasculature may be interrupted; this may result in the limbal tissue (including limbal stem cells) suffering necrosis, for example. Delivering a high concentration of topical oxygen post-injury may facilitate preserving “at-risk” tissue for a prolonged period of time. In some cases, topical oxygen may penetrate through the corneal and/or limbal tissue to a depth that may be adequate to oxygenate limbal stem cells.

In general, if blood flow to the ocular area becomes interrupted due to trauma, for example, increasing oxygen delivery to the limbal cells, or other cells, may be extremely important in preserving those cells. In some cases, blood supply to the ocular region may be provided by retinal vasculature, an oxygen emulsion in accordance with various embodiments may increase the partial pressure of oxygen at the surface of the eye and potentially reach other ocular region cells (e.g., corneal epithelial cells and possibly the limbus region), since some embodiments may increase the driving pressure for oxygen to diffuse across tissues.

Some embodiments may utilize systems and/or devices such as shown in system 100-a of FIG. 1A, system 100-b of FIG. 1B, and/or devices 200 of FIG. 2A, FIG. 2B, FIG. 2C, FIG. 2D, FIG. 2E, FIG. 2F, FIG. 2G, FIG. 2H, FIG. 2I, and/or FIG. 2J for delivering oxygen topically to the cornea and outer tissues (including the eye lids and marginal structure) through a highly-saturated emulsion. Field application may include both traumatic injury (as noted above) as well as potentially in the treatment of degenerative eye disorders (cataracts, dry eye syndrome, etc.).

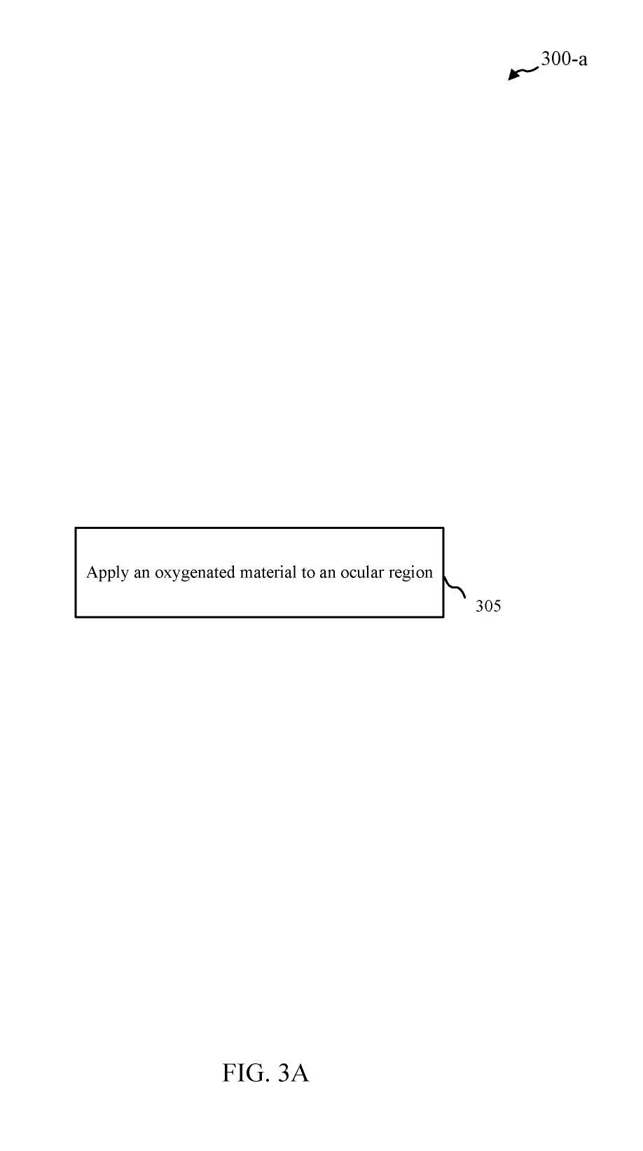

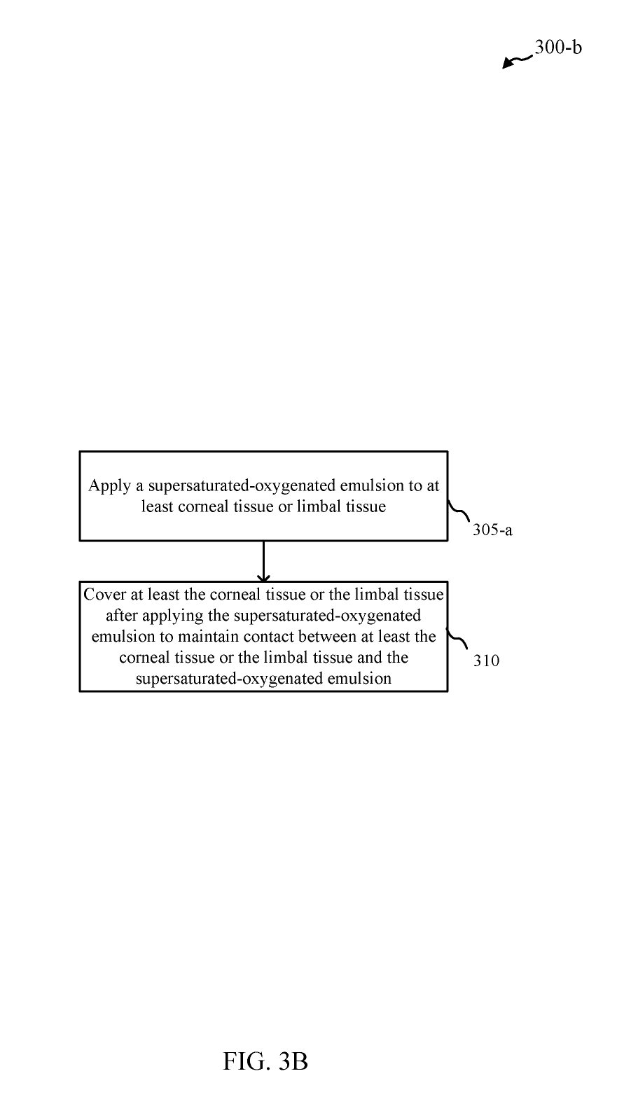

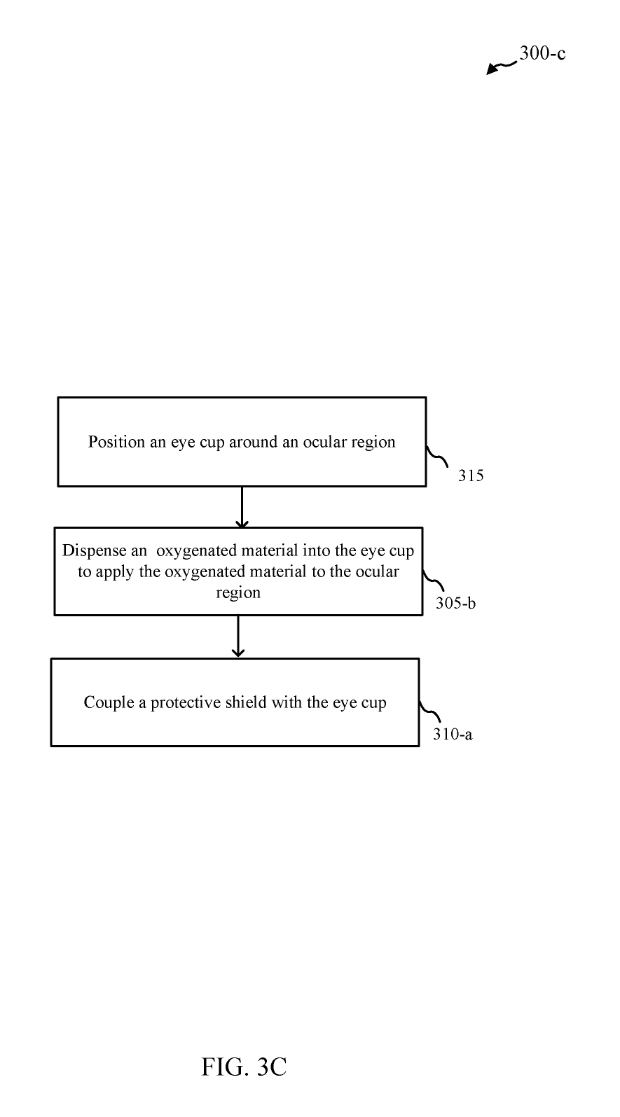

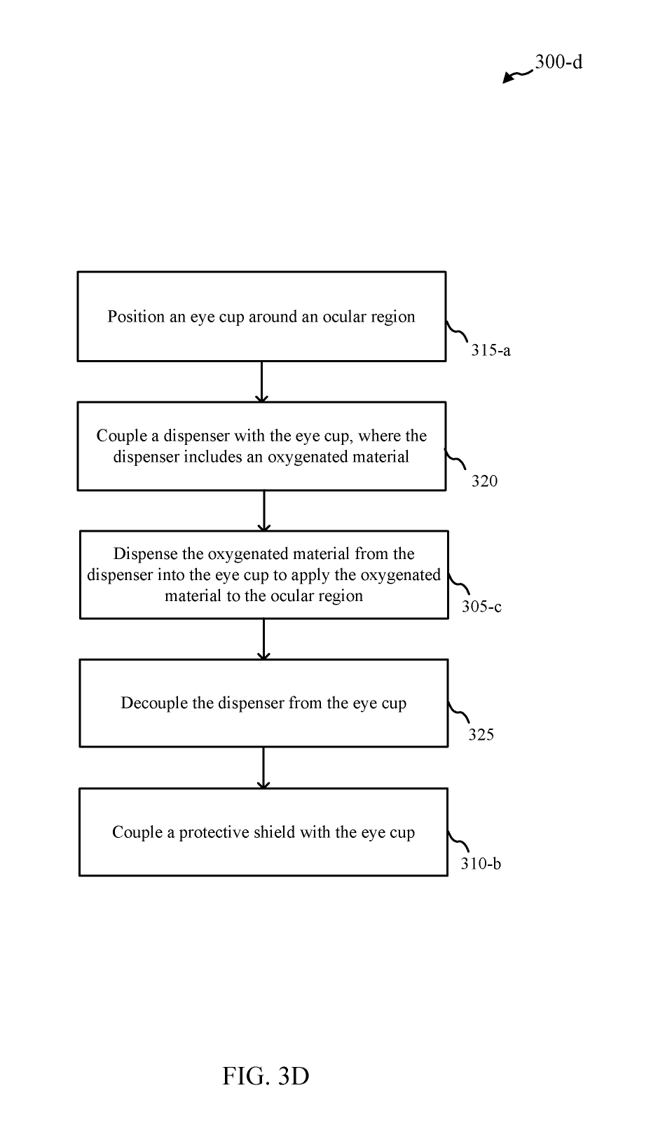

Turning now to FIG. 3A, a flow diagram of an ocular region treatment method 300-a is shown in accordance with various embodiments. Method 300-a may be implemented utilizing a variety of systems and/or devices such as those shown and/or described with respect to FIG. 1A, FIG. 1B, FIG. 2A, FIG. 2B, FIG. 2C, FIG. 2D, FIG. 2E, FIG. 2F, FIG. 2G, FIG. 2H, FIG. 2I, and/or FIG. 2J. In FIG. 3A, the specific selection of steps shown and the order in which they are shown is intended merely to be illustrative. It is possible for certain steps to be performed in alternative orders, for certain steps to be omitted, and for certain additional steps to be added according to different embodiments of the invention. Some but not all of these variants are noted in the description that follows.

At block 305, an oxygenated material may be applied to an ocular region. The ocular region may include at least corneal tissue or limbal tissue. In some cases, the ocular region includes ocular adnexal tissue.

The oxygenated material may include at least an oxygenated emulsion, an oxygenated ointment, an oxygenated hydrogel, or an oxygenated liquid. The oxygenated emulsion, the oxygenated ointment, the oxygenated hydrogel, or the oxygenated liquid may include at least a supersaturated-oxygenated emulsion, a supersaturated-oxygenated ointment, a supersaturated-oxygenated hydrogel, or a supersaturated-oxygenated liquid.

In some embodiments of method 300-a, the oxygenated material includes perfluorocarbon. The perfluorocarbon may include perfluorodecalin. The oxygenated material may include at least an antibiotic, an anti-inflammatory, or an anesthetic.

In some embodiments of method 300-a, the oxygenated material is configured to produce a partial pressure of O₂ above that which exists at ambient atmospheric pressure when applied to the ocular region.

Some embodiments of the method 300-a may further include positioning an eye cup around the ocular region. The oxygenated material may be dispensed into the eye cup as part of the process of applying the oxygenated material to the ocular region. Method 300-a may include coupling a protective shield with the eye cup. In some embodiments, dispensing the oxygenated material into the eye cup includes dispensing the oxygenated material through at a side aperture of the eye cup. In some embodiments, dispensing the oxygenated material into the eye cup includes dispensing the oxygenated material through a transparent layer coupled with a top aperture of the eye cup. Some embodiments of method 300-a include coupling a dispenser with the eye cup to dispense the oxygenated material into the eye cup. In some embodiments, the dispenser may be decoupled from the eye cup; protective shield may be coupled with the eye cup. Some embodiments include sealing the eye cup around the ocular region.

Some embodiments of the method 300-a include covering the ocular region after applying the oxygenated material to maintain contact between the ocular region and the oxygenated material.

Applying the oxygenated material to the ocular region may improve healing of the ocular region in some cases. Applying the oxygenated material to the ocular region may facilitate healing of a trauma to the ocular region. Applying the oxygenated material to the ocular region may facilitate preserving tissues in the ocular region. Applying the oxygenated material to the ocular region may occur after a trauma to the ocular region. Applying the oxygenated material to the ocular region may facilitate treatment of at least a disorder of the ocular region, symptoms from the disorder of the ocular region, or a side-effect of a medication.

Some embodiments of the method 300-a include identifying at least a trauma to the ocular region or a disorder of the ocular region before applying the oxygenated material to the ocular region. Applying the oxygenated material to the ocular region may at least replace a physiological process of the ocular region or augment the physiological process of the ocular region.

FIG. 3B shows a flow diagram of an ocular region treatment method 300-b in accordance with various embodiments. Method 300-b may be implemented utilizing a variety of systems and/or devices such as those shown and/or described with respect to FIG. 1A, FIG. 1B, FIG. 2A, FIG. 2B, FIG. 2C, FIG. 2D, FIG. 2E, FIG. 2F, FIG. 2G, FIG. 2H, FIG. 2I, and/or FIG. 2J. In FIG. 3B, the specific selection of steps shown and the order in which they are shown is intended merely to be illustrative. It is possible for certain steps to be performed in alternative orders, for certain steps to be omitted, and for certain additional steps to be added according to different embodiments of the invention. Some but not all of these variants are noted in the description that follows. Method 300-b may be an example of method 300-a.

At block 305-a, a supersaturated oxygenated emulsion may be applied to at least corneal tissue or limbal tissue. At block 310, at least the corneal tissue or the limbal tissue may be covered after applying the supersaturated-oxygenated emulsion to maintain contact between at least the corneal tissue or the limbal tissue and the supersaturated-oxygenated emulsion.

FIG. 3C shows a flow diagram of an ocular region treatment method 300-c in accordance with various embodiments. Method 300-c may be implemented utilizing a variety of systems and/or devices such as those shown and/or described with respect to FIG. 1A, FIG. 1B, FIG. 2A, FIG. 2B, FIG. 2C, FIG. 2D, FIG. 2E, FIG. 2F, FIG. 2G, FIG. 2H, FIG. 2I, and/or FIG. 2J. In FIG. 3C, the specific selection of steps shown and the order in which they are shown is intended merely to be illustrative. It is possible for certain steps to be performed in alternative orders, for certain steps to be omitted, and for certain additional steps to be added according to different embodiments of the invention. Some but not all of these variants are noted in the description that follows. Method 300-c may be an example of method 300-a and/or method 300-b.

At block 315, an eye cup may be positioned around an ocular region. At block 305-b, an oxygenated material may be dispensed into the eye cup to apply the oxygenated material to the ocular region. At block 310-a, a protective shield may be coupled with the eye cup.

FIG. 3D shows a flow diagram of an ocular region treatment method 300d in accordance with various embodiments. Method 300d may be implemented utilizing a variety of systems and/or devices such as those shown and/or described with respect to FIG. 1A, FIG. 1B, FIG. 2A, FIG. 2B, FIG. 2C, FIG. 2D, FIG. 2E, FIG. 2F, FIG. 2G, FIG. 2H, FIG. 2I, and/or FIG. 2J. In FIG. 3D, the specific selection of steps shown and the order in which they are shown is intended merely to be illustrative. It is possible for certain steps to be performed in alternative orders, for certain steps to be omitted, and for certain additional steps to be added according to different embodiments of the invention. Some but not all of these variants are noted in the description that follows. Method 300d may be an example of method 300-a, method 300-b, and/or method 300-c.

At block 315-a, an eye cup may be positioned around an ocular region. At block 320, a dispenser may be coupled with the eye cup, where the dispenser includes an oxygenated material. At block 305-c, the oxygenated material may be dispensed from the dispenser into the eye cup to apply the oxygenated material to the ocular region. At block 325, the dispenser may be decoupled from the eye cup. At block 310-b, a protective shield may be coupled with the eye cup.

These embodiments may not capture the full extent of combination and permutations of materials and process equipment. However, they may demonstrate the range of applicability of the method, devices, and/or systems. The different embodiments may utilize more or less stages than those described.

It should be noted that the methods, systems and devices discussed above are intended merely to be examples. It must be stressed that various embodiments may omit, substitute, or add various procedures or components as appropriate. For instance, it should be appreciated that, in alternative embodiments, the methods may be performed in an order different from that described, and that various stages may be added, omitted or combined. Also, features described with respect to certain embodiments may be combined in various other embodiments. Different aspects and elements of the embodiments may be combined in a similar manner. Also, it should be emphasized that technology evolves and, thus, many of the elements are exemplary in nature and should not be interpreted to limit the scope of the embodiments.

Specific details are given in the description to provide a thorough understanding of the embodiments. However, it will be understood by one of ordinary skill in the art that the embodiments may be practiced without these specific details. For example, well-known circuits, processes, algorithms, structures, and techniques have been shown without unnecessary detail in order to avoid obscuring the embodiments.

Also, it is noted that the embodiments may be described as a process which may be depicted as a flow diagram or block diagram or as stages. Although each may describe the operations as a sequential process, many of the operations can be performed in parallel or concurrently. In addition, the order of the operations may be rearranged. A process may have additional stages not included in the figure.

Having described several embodiments, it will be recognized by those of skill in the art that various modifications, alternative constructions, and equivalents may be used without departing from the spirit of the different embodiments. For example, the above elements may merely be a component of a larger system, wherein other rules may take precedence over or otherwise modify the application of the different embodiments. Also, a number of stages may be undertaken before, during, or after the above elements are considered. Accordingly, the above description should not be taken as limiting the scope of the different embodiments.

1.-24. (canceled)

25. An ocular region treatment system comprising:
an eye cup configured to surround an ocular region; and
a dispenser configured to dispense an oxygenated material to the ocular region.

26. The system of claim 25, wherein the dispenser is configured to couple with the eye cup.

27. The system of claim 25, wherein the dispenser includes an aerosol can containing the oxygenated material.

28. The system of claim 26, wherein at least the dispenser is configured to decouple from the eye cup or the aerosol can is configured to decouple from the dispenser.

29. The system of claim 25, further comprising a protective shield configured to couple with the eye cup to create a closed space to contain the dispensed oxygenated material around the ocular region.

30. The system of claim 25, wherein the oxygenated material includes at least an oxygenated emulsion, an oxygenated ointment, an oxygenated hydrogel, or an oxygenated liquid.

31. The system of claim 30, wherein the oxygenated liquid, the oxygenated ointment, the oxygenated hydrogel, or the oxygenated emulsion includes at least a supersaturated-oxygenated emulsion, a supersaturated-oxygenated ointment, supersaturated-oxygenated hydrogel, or a supersaturated-oxygenated liquid.

32. The system of claim 25, wherein the oxygenated material includes perfluorodecalin.

33. An ocular region treatment device comprising:
a rigid base with at least a top aperture and a bottom aperture, wherein the bottom aperture is configured to encompass an ocular region for treatment.

34. The device of claim 33, further comprising a seal coupled with the rigid base around the bottom aperture.

35. The device of claim 34, wherein the seal includes a rubber gasket.

36. The device of claim 35, further comprising a protective shield configured to couple with and to decouple from the rigid base.

37. The device of claim 36, further comprising a transparent layer configured to cover the top aperture of the rigid base.

38. The device of claim 37, wherein the transparent layer includes one or more apertures configured to allow for an oxygenated material to be introduced into the device.

39. The device of claim 36, wherein the protective shield includes one or more protrusions configured to facilitate the coupling and the decoupling of the protective shield from the rigid base.

40. The device of claim 36, wherein the protective shield includes one or more apertures configured to facilitate the coupling and the decoupling of the protective shield from the rigid base.

41. The device of claim 36, wherein the protective shield includes a semi-rigid material.

42. The device of claim 33, wherein the rigid base includes one or more side apertures configured to allow for an oxygenated material to be introduced into the device.

43. The device of claim 33, wherein the protective shield includes one or more apertures configured to allow for an oxygenated material to be introduced into the device.

44. The device of claim 34, wherein the seal includes one or more adhesives to facilitate sealing around the ocular region.
